(19) United States
(12) Patent Application Publication
(10) Pub. No.: US 2010/0055059 A1
(43) Pub. Date: Mar. 4, 2010

(54) ASSOCIATION OF COMPOUNDS INHIBITING MELANOGENESIS AND USE THEREOF IN COSMETICS AND DERMATOLOGY

(75) Inventors: Marc Criton, Montpellier (FR); Veronique Lemellay Hamon, Montpellier (FR); Yves Leblond, Orgeval (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: LABORATOIRES MAYOLY SPINDLER, CHATOU (FR)

(21) Appl. No.: 12/522,261

(22) PCT Filed: Jan. 15, 2008
(86) PCT No.: PCT/FR08/00040

§ 371 Date: Nov. 4, 2009

(30) Foreign Application Priority Data
Jan. 16, 2007 (FR) 07 00279

(51) Int. Cl. A61K 8/64; A61K 38/06; A61Q 19/02

(52) U.S. Cl. 424/62; 514/18

(57) ABSTRACT

The invention relates to an association including at least : a MC1-R receptor inhibitor; a tyrosinase inhibitor derived from vitamin C; an inhibitor of the transfer of melanosomes to the keratinocytes; the invention also relates to the use thereof in cosmetics and dermatology for preparing whitening and/or bleaching depigmentation compositions.

The subject of the invention is a novel combination of melanogenesis-inhibiting compounds and the uses thereof in cosmetics and in dermatology for preparing whitening and/or lightening depigmentation compositions.

Lightening products, which are prized by Asian countries where whiteness of the skin is a real aesthetic requirement, but also by populations in the West where a homogeneous complexion is a sign of a healthy body, are a real phenomenon of society. The dermatology and cosmetics sectors have been able to meet this need by proposing lightening or whitening depigmentation products intended to promote elimination of pigmentary spots (sun spots, freckles, senescence spots) or to lighten the complexion.

Skin and follicular pigmentation is the result of the exposure of melanin at the surface of the skin and of the hair follicle. Melanogenesis is carried out specifically by the melanocytes, dendritic cells present in the basal layer of the epidermis, which put out branches for contact with the keratinocytes. The newly synthesized melanin is transferred from the melanocyte dendrites to the keratinocytes, which ultimately expose the melanin at the surface of the epidermis, thus providing uniform coloration of the epidermis.

The synthesis of melanins (pheomelanins, rich in sulfur, giving an orangey color; eumelanins, conferring a brown color) is carried out in the melanosomes, which are melanocyte-specific lysosome-like organelles, by a complex enzymatic process. Three enzymes located on the internal face of the melanosomal membrane are successively involved in melanogenesis: tyrosinase, TRP-2 (tyrosinase-related protein-2) and TRP-1 (tyrosinase-related protein-1). Tyrosine, a precursor for melanin synthesis, is hydroxylated to Dopa (dihydroxyphenylalanine) and then oxidized to dopaquinone, these two conversions being due to the action of tyrosinase.

At this stage, the melanin synthesis can be oriented toward pheomelanin (orangey-yellow melanin) which is encountered in blond individuals or redheads, or toward eumelanin (dark brown melanin) which is encountered in individuals with dark pigmentation. Eumelanin results from the polymerization of dopaquinone so as to give leukodopachrome and then dopachrome. The latter is in turn converted either to 5,6-dihydroxyindole (DHI) or to 5,6-dihydroxyindole-2-carboxylic acid (DHICA) under the action of TRP-2. At this level, the synthesis of eumelanin can be carried out according to two pathways. DHI is oxidized, under the action of tyrosinase or of a peroxidase, to indole-5,6-quinone, while DHICA, under the action of TRP-1, gives 5,6-dihydroindole-2-carboxylic acid. The indole-5,6-quinone and the 5,6-dihydroindole-2-carboxylic acid polymerize so as to form melanochromes and then eumelanin.

The synthesis of pheomelanin involves the formation of sulfur compounds (cysteinyl-DOPA) subsequent to the action on dopaquinone of glutathione and of cysteine. The cysteinyl-DOPA is converted to alanylhydroxybenzothiazine, and then to pheomelanin.

The molecular mechanisms that regulate melanocytes and melanin production are relatively poorly elucidated. The studies by Y. Yada have shown that, under the effect of solar radiation of UVB type, human keratinocytes produce and secrete endothelin peptide hormone which exerts a paracrine effect on the melanocytes (Imokawa G. et al., J. Invest. Dermatol., 105: 32-37, 1995). Endothelin activates a G protein-coupled membrane receptor (ETR) inducing melanocyte proliferation, and transcription of the genes encoding tyrosinase and the ETR. Similarly, in response to UV radiation, keratinocytes and melanocytes secrete the αMSH peptide (melanocortin-stimulating hormone) which regulates melanocyte pigmentation activity. To do this, αMSH binds to MC-R (the melanocortin receptor), inducing activation of the cAMP/PKA transduction pathway, or even of the ser/thr kinase PKC, resulting in de novo synthesis of tyrosinase and in eumelanin synthesis. PKCβ appears to directly activate tyrosinase by phosphorylation of the cytoplasmic domain thereof (Park et al., J. Biol. Chem., 268: 11742-11749, 1993). αMSH also appears to facilitate the transfer of melanin to keratinocytes by stimulating melanocyte dendricity (Hunt et al., J. Cell. Sci., 107: 205-211, 1994).

In addition to the desire, for certain individuals or populations, to obtain and to conserve a light complexion, there is also, for many, the problem of the prevention and treatment of localized hyper-pigmentations, in the form of marks. A localized hyper-pigmentation of the skin may be of endogenous origin, as is the case with freckles, which are common in individuals with a light complexion. It may also be the result of exposure to UV radiation. An increase in freckles, which become darker in color, is observed under the effect of UV radiation. The appearance of cutaneous hyperpigmentation spots is also noted in areas subjected to irritation (insect bite, slowly healing wound, eczema, etc.). The hormonal factor is responsible for regional hyperpigmentations due to melanocyte hyperactivity, such as idiopathic melasmas occurring during pregnancy (pregnancy mask) or oestro-progestative contraception. Similarly, pigmentary spots due to benign melanocyte hyperactivity and proliferation often appear in elderly individuals (senile lentigo).

The substances known for their depigmenting properties can act according to one of the following mechanisms:
on the viability of the epidermal and/or follicular melanocytes where melanogenesis takes place,
by interfering with one of the stages of melanin biosynthesis, or by inhibiting one of the enzymes involved in melanogenesis, or by intercalating as a structural analogue of one of the chemical compounds of the melanin synthesis chain,
by interfering in the transfer of melanin from melanocytes to keratinocytes.

The substances most widely used are mostly inhibitors of tyrosinase activity. Mention may be made of phenolic derivatives. These derivatives have a chemical structure comparable to that of tyrosine or of dopa and serve as a substrate for tyrosinase (competitive inhibition). Hydroquinone and derivatives thereof are found in this family. However, these products exhibit considerable cytotoxicity capable of causing irreversible depigmentations, and the use of hydroquinone in cosmetic products has been prohibited by European regulation (Dir. 2000/6/BC).

Various other substances are proposed as a depigmenting agent. Some exhibit good local tolerance but also a low efficacy: vitamin C, arbutin (hydroquinone β-D-gluconopyranoside), niacinamide, which acts on the transfer of melanosomes from melanocytes to keratinocytes (Hakozaki T. et al., British Journal of Dermatology, 147: 20-31, 2002), plant extracts, in particular soybean extracts (Paine C. et al., Journal of Investigative Dermatology, 116, 4: 587-595, 2001), which inhibit the activity of the receptor PAR-2 (protease-activated receptor 2) expressed by the keratinocytes.

The complexity of the mechanisms involved in melanin synthesis makes it difficult to control the pigmentation of the skin under conditions which are satisfactory for user health (Briganti S. et al., Pigment Cell Res., 16: 101-110, 2003).

It has therefore been sought to develop compositions capable of preventing and/or treating the appearance of hyperpigmentation of the skin and/or of the superficial body growths, whether it is hyperpigmentation of endogenous origin or of exogenous origin (UV, skin irritation, hormonal). It has also been sought to develop compositions which are not toxic and the action of which is reversible.

In particular, it has been sought to obtain good efficacy with a view to preventing or treating regional hyperpigmentations due to melanocyte hyperactivity, such as:
idiopathic melasmas, occurring during pregnancy, also called pregnancy mask or chloasma, or melasmas which are the consequence of oestroprogestative contraception;
localized hyperpigmentations caused by benign melanocyte hyperactivity and proliferation, such as freckles, sun spots or senile pigmentation spots, known as actinic lentigo;
accidental hyperpigmentations or depigmentations, possibly due to photosensitization or to post-lesional healing, for instance in areas subjected to an irritation (insect bite, slowly healing wound, eczema, etc.), and also certain forms of leukoderma such as vitiligo.

It has also been sought to develop compositions having the property of whitening the skin and/or of lightening the complexion and/or of making the complexion uniform and/or of making the complexion homogeneous.

The present invention concerns a combination comprising at least three compounds:
an MC1-R receptor antagonist,
a vitamin-C-derived tyrosinase inhibitor,
an inhibitor of melanosome transfer to keratinocytes.

Surprisingly, it has been noted that the combination of three compounds as defined above results in an inhibition that is greater than the isolated action of these 3 active agents. In addition, it is devoid of toxic effects and the combined action of the three active agents is reversible.

For the purpose of the present invention, the term “MC1-R receptor antagonist” is intended to mean a compound capable of binding to the melanotropin cellular receptors (MC1-R) present on the melanocyte membrane, of blocking the binding of αMSH (melanocortin stimulating hormone), a ligand specific for MC1-R, and of inhibiting the activation of MC1-R by αMSH. Two endogenous MCR antagonists exist, including the Agouti protein, which has a strong affinity for MC1-R and is involved in the regulation of skin pigmentation (Suzuki I et al., J. Invest Dermatol, 108: 838-842, 1997). Among the MC1-R receptor antagonists known to those skilled in the art, mention may in particular be made of an oligopeptide discovered by the Institut Européen de Biologie cellulaire (IEB) [European Cell Biology Institute], Melanostatine®5 (INCI name: nonapeptide-1), sold by the company Unipex. This oligopeptide has an affinity for MC1-R receptors and specifically and reversibly inhibits melanogenesis by decreasing the synthesis and the excessive production of melanin pigments. This active agent is, moreover, devoid of toxic effects. Mention may also be made of the lipoamino acid undecylenoyl phenylalanine sold by the company SEPPIC under the name Sepiwhite MSH®.

For the purpose of the present invention, the term “vitamin-C-derived tyrosinase inhibitor” is intended to mean a compound chosen from ascorbic acid esters, for instance ascorbic acid 2-glucoside (INCI name: Ascorbyl Glucoside), 2-O-alpha-D-glucopyranosyl-6-O-hexa-decanoyl-L-ascorbic acid, ascorbyl 6-palmitate, or the magnesium or sodium salt of ascorbic acid 2-phosphate. Preferably, ascorbic acid 2-glucoside, which is sold in particular by the company DKSH under the trade mark AA-2G®, is used.

For the purpose of the present invention, the term “inhibitor of melanosome transfer” is intended to mean a compound capable of inhibiting melanosome transfer to keratinocytes (Greatens A. et al., Exp Dermatol, 14: 498-508, 2005; Hakozaki T. et al., Br. J Dermatol, 147: 20-31, 2002). In particular, this definition is intended to mean a compound such as nicotinamide (INCI name: niacinamide), or vitamin PP, which is one of the two forms of vitamin B3 and which is sold by the company MERCK.

The present invention therefore concerns a combination constituted of at least three active agents devoid of toxic effects, which act on melanogenesis via three different mechanisms.

Given the complexity of the mechanisms of melanogenesis, it was expected that the combination of these three active agents would have an effect that was not much greater than that of the best of the three. Surprisingly, it was noted that the combination of these three active agents led to an inhibition of melanogenesis that was much greater than the expected result, whether it was endogenous melanogenesis or melanogenesis caused by an external agent such as UV radiation, and that, in addition, this inhibition was completely reversible.

A subject of the present invention is also cosmetic and/or dermatological compositions comprising this combination in a cosmetically or dermatologically acceptable carrier, compatible with application to the skin, body hairs or head hair.

A subject of the invention is also the use of this combination in a cosmetic composition or for the preparation of a dermatological composition, for the purpose of preventing or treating regional hyper-pigmentations, and/or of whitening the skin, and/or of lightening the complexion, and/or of making the complexion uniform, and/or of making the complexion homogeneous.

In the compositions of the invention, the three compounds are present in an amount preferentially included in a range of from:
0.01% to 5% for the inhibitor of melanosome transfer, in particular nicotinamide,
0.01% to 5% for the vitamin-C-derived tyrosinase inhibitor, in particular for ascorbic acid 2-glucoside,
0.1 to 40 ppm for the MCL-R receptor antagonist, in particular Melanostatine®5, preferentially 2 to 4 ppm.

The compositions according to the invention are preferably suitable for topical application to the skin. The cosmetic and/or dermatological compositions of the invention can be used for preventive or curative purposes.

The compositions according to the invention may also comprise UVA or UVB screens, conventionally used in day care or make-up formulations in cosmetics or in dermatology.

They may also comprise additional active agents such as exfoliant active agents, which also have a lightening effect on the skin: for example, mention may be made of alpha- and beta-hydroxy acids.

The compositions of the invention may be in any of the galenical forms normally used for topical application, and in particular in the form of an aqueous solution, an aqueous-alcoholic solution or an oily solution. They may be in the form of a water-in-oil or oil-in-water emulsion, a multiple emulsion, a dispersion of nanoparticles or of lipid vesicles of the liposome type, an aqueous gel, an oily gel, or a liquid, pasty or solid anhydrous product. This cosmetic and/or dermatological composition may be constituted of a formulation of the type: lotion, gel, cream, foam, ointment, patch, mask, stick, shampoo, conditioner, make-up product.

In a known manner, the composition of the invention may also contain adjuvants that are customary in the cosmetics or dermatology field, such as, for example, hydrophilic or lipophilic gelling agents, emulsifiers, preservatives, antioxidants, fillers, solvents, fragrances, pigments or dyes. It may also contain one or more other active agents, which may be present in the same phase as the combination of the invention or, if it is a composition comprising several phases, in another phase of the composition. Among the other active agents that can be used in the compositions of the invention, mention may be made of moisturizing agents, such as glycerol, anti-aging agents, such as anti-wrinkle agents, for instance alpha-hydroxy acids, 7-hydroxy-DHEA or retinol. As oils that can be used in the compositions of the invention, mention may be made of mineral oils (liquid petroleum jelly, paraffin oil), plant oils (avocado oil, soybean oil), oils of animal origin (lanolin), silicone oils (cyclomethicone) and synthetic oils. The composition may also contain other fatty substances, such as fatty alcohols or waxes (carnauba wax, beeswax).

As emulsifiers, mention may be made, in a known manner, of: fatty acid esters of polyethylene glycol, fatty acid esters of glycerol.

### BIOLOGICAL EVALUATION

The evaluations regarding melanogenesis were carried out in vitro on melanized reconstructed epidermis, optionally subjected to UV irradiation.

Moreover, we showed that the use of this combination does not result in a total block of the mechanisms enabling melanocytes to produce melanin possibly in response to solar irradiation, for example. Thus, we demonstrated the reversibility of the inhibitory effects of the composition thereof using a study carried out on models of human melanocytes cultured in monolayer.

### FIGURES

FIG. 1: Reversibility of the melano-inhibitory activity of a complex of active agents containing the products MS-A, MS-N and MS-Mel in a model of human melanocytes in monolayer culture having been subjected to UVB irradiation.

### I—Study of the Reversibility of the Melano-Inhibitory Effect of the Combination in a Model of Normal Human Melanocytes Cultured in Monolayer

The objective of this study was to evaluate the reversibility of the melano-inhibitory effect of the complex containing the products MS-A, MS-N and MS-Mel, in a model of normal human melanocytes in monolayer culture.

### I. 1—Products Tested

The products AA-2G®, Nicotinamide® and Melanostatine® 5 are respectively noted MS-A, MS-N and MS-Mel.

### I. 2—Test System

Normal human melanocytes were obtained from a foreskin of a 4-year-old individual. To carry out the tests, these cells were cultured until confluent monolayers were obtained.

### I. 3—Reference Inhibitor

The reference inhibitor used in this study was kojic acid at 250 μM.

I. 4—Incubation of the Cells with the Test Products

The melanocytes were incubated for 72 hours at 37° C., in a humid atmosphere and 5% CO₂, in the absence (control) or in the presence of kojic acid or of the complex of test active agents containing MS-A at 2.5×10⁻⁴ M, MS-N at 2.5×10⁻⁴ M and MS-Mel at 10⁻⁴ M. At the end of this first incubation period, the reference product and the complex of test active agents were removed from the incubation media, and the cells were incubated for a further 72 hours in the presence of culture medium alone. Every 24 hours, the cells were then irradiated with UVB radiation at 0.05 J/cm², or not irradiated.

### I. 5—Evaluation of the Effects

### I. 5.1—Assaying of Melanin

At the end of the incubation period, the intracellular content of melanin was quantified in the cell lysates by spectrophotometric measurement at 405 nm.

### I. 5.2—Assaying of Proteins

At the end of the incubation period, the proteins contained in the cell lysates were quantified by the Bradford spectrocolorimetric method.

### I. 6—Results

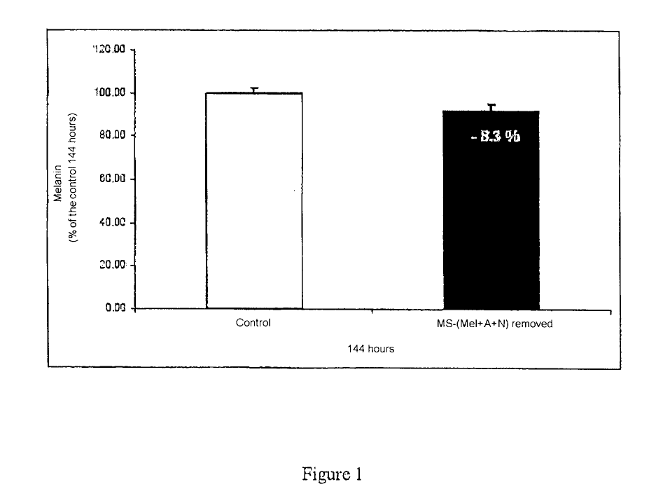

After a first incubation period in the presence of the complex, which resulted in significant inhibition of melanogenesis (−24.6% at T72h, p<0.05), the cells kept their ability to produce melanin when the products were removed from the culture medium, this being the case without UV irradiation or after UV irradiation. Specifically, the inhibition is now only 9.6% and 8.3% without UV irradiation or after UV irradiation, respectively (FIG. 1).

The present study made it possible to show that the use of a depigmenting complex containing the products MS-A, MS-N and MS-Mel did not lead to complete and nonspecific blocking of the mechanisms involved in melanogenesis. In fact, its melano-inhibitory activity was entirely reversible, since cells placed in the presence of this complex for 72 hours kept their ability to produce melanin when the latter was removed from the incubation media.

### FORMULATION

In the examples, the percentages are percentages by weight.

### Example 1

### Lightening Care Cream

| | |
|---|---|
| Arlacel ® 165 | 5.00 |
| Beeswax | 5.00 |
| Liquid paraffin oil | 5.00 |
| Cetyl alcohol | 3.00 |
| AA-2G ® | 2.00 |
| Nicotinamide | 2.00 |
| Emulgin ® B2 | 2.00 |
| Sepigel ® 305 | 1.60 |
| Melanostatine ® 5 | 0.40* |
| Methylparaben | 0.30 |
| Propylparaben | 0.20 |
| Thromethamine | qs pH = 6.2 |
| Demineralized water | qs 100 |

(*i.e. 4 ppm in the cream)

### Example 2

### Lotion

| | |
|---|---|
| Glycerol | 3.00 |
| AA-2G ® | 2.00 |
| Nicotinamide | 2.00 |
| Pluronic ® PE 6400 | 2.00 |
| Cosmocil CQ ® | 0.50 |
| Melanostatine ® 5 | 0.40* |
| Fragrance | 0.20 |
| Thromethamine | qs pH = 6.4 |
| Demineralized water | qs 100 |

(*i.e. 4 ppm in the lotion)

### Example 3

### “Mature Skin” Care Cream

| | |
|---|---|
| Arlacel ® 165 | 5.00 |
| Beeswax | 5.00 |
| Liquid paraffin oil | 6.00 |
| Cetyl alcohol | 3.00 |
| AA-2G ® | 2.00 |
| Nicotinamide | 2.00 |
| Gatuline ® RC | 2.00 |
| Emulgin ® B2 | 2.00 |
| Nuteline ® C | 2.00 |
| Sepigel ® 305 | 1.80 |
| CM GLUCAN P | 1.00 |
| MELANOSTATINE ® 5 | 0.40* |
| Methylparaben | 0.30 |
| Propylparaben | 0.20 |
| α-Lupaline ® | 0.10 |
| Thromethamine | qs pH = 6.3 |
| Demineralized water | qs 100 |

(*i.e. 4 ppm in the cream)

1. A combination comprising at least one MC1-R receptor antagonist, one vitamin-C-derived tyrosinase inhibitor and one inhibitor of melanosome transfer to keratinocytes, wherein said MC1-R receptor antagonist is MELANOSTATINE® 5.

2. The combination as claimed in claim 1, wherein the vitamin-C-derived tyrosinase inhibitor is ascorbic acid 2-glucoside.

3. The combination as claimed in claim 1, wherein the inhibitor of melanosome transfer to keratinocytes is nicotinamide.

4. A cosmetic and/or dermatological composition comprising the combination as claimed in claim 1 in a cosmetically or dermatologically acceptable carrier.

5. The cosmetic and/or dermatological composition as claimed in claim 4, wherein it comprises:
0.01% to 5% of melanosome transfer inhibitor,
0.01% to 5% of vitamin-C-derived tyrosinase inhibitor,
0.1 to 40 ppm of MC1-R receptor antagonist.

6. The cosmetic and/or dermatological composition as claimed in claim 5, wherein it comprises:
0.01% to 5% of nicotinamide,
0.01% to 5% of ascorbic acid 2-glucoside,
0.1 to 40 ppm of MELANOSTATINE®.

7. A composition for preventing and/or treating the appearance of hyperpigmentation of the skin and/or of superficial body growths comprising the cosmetic and/or dermatological composition as claimed in claim 4.

8. A method for preventing or treating the appearance of hyperpigmentation of the skin and/or of superficial body growths comprising applying to said appearance the combination as claimed in claim 1.

9. The composition as claimed in claim 7, for whitening the skin and/or lightening the complexion and/or making the complexion uniform and/or making the complexion homogeneous.

10. The composition as claimed in claim 7, for preventing or treating regional hyperpigmentations due to melanocyte hyperactivity, selected from:
idiopathic melasmas, or melasmas which are the consequence of oestroprogestative contraception;
localized hyperpigmentations caused by benign melanocyte hyperactivity and proliferation; and
accidental hyperpigmentations or depigmentations.
